(19) United States
(12) Patent Application Publication
(10) Pub. No.: US 2016/0040320 A1
(43) Pub. Date: Feb. 11, 2016

(54) METHOD OF MANUFACTURING POROUS THREE-DIMENSIONAL MICRO/NANOFIBROUS SCAFFOLD USING ELECTROHYDRODYNAMIC PROCESS AND POROUS THREE-DIMENSIONAL MICRO/NANOFIBROUS SCAFFOLD MANUFACTURED THEREBY

(71) Applicant: RESEARCH & BUSINESS FOUNDATION SUNGKYUNKWAN UNIVERSITY, Suwon-si (KR)

(72) Inventors: GeunHyung KIM, Seongnam-si (KR); Minseong KIM, Sinan-gun (KR)

(73) Assignee: RESEARCH & BUSINESS FOUNDATION SUNGKYUNKWAN UNIVERSITY, Suwon-si (KR)

(21) Appl. No.: 14/750,639

(22) Filed: Jun. 25, 2015

(30) Foreign Application Priority Data
Aug. 7, 2014 (KR) 10-2014-0101477

(51) Int. Cl. D01D 5/00; D01D 5/06; A61L 27/18

(52) U.S. Cl. CPC D01D 5/003; A61L 27/18; D01D 5/06; D10B 2331/041; D10B 2509/00

(57) ABSTRACT

Provided are a method of manufacturing a porous three-dimensional microfiber, more particularly, a micro/nanofibrous scaffold using an electrohydrodynamic (EHD) process and a porous three-dimensional micro/nanofibrous scaffold manufactured thereby.

According to the method, the porous three-dimensional micro/nanofibrous scaffold, in which a pore structure is controlled by a process of phase changing an initial jet manufactured through the EHD process from a gas phase to a liquid phase using a liquid collector having a low surface tension, may be manufactured.

In addition, the scaffold manufactured by the manufacturing method according to the present invention has a very similar structure to an extracellular matrix (ECM), and is considerably improved in a pore structure and porosity, thereby increasing an cell attached area and enabling cells to permeate into the scaffold, and thus can provide an optimal environment for in vitro or in vivo cell growth to proliferate the cells.

### STATEMENT REGARDING GOVERNMENT RIGHTS

This invention was made with government supports of the Republic of Korea under Contract Nos. A120942 and 2012R1A2A01017435 awarded by Korean Ministry of Health and Welfare, and Ministry of Education and Science Technology, respectively. The government has certain rights in the invention.

### CROSS-REFERENCE TO RELATED APPLICATION

This application claims priority to and the benefit under 35 U.S.C. §119(a) of Korean Patent Application No. 2014-0101477, filed on Aug. 7, 2014, the disclosure of which is incorporated herein by reference in its entirety.

### BACKGROUND

1. Field of the Invention

The present invention relates to a method of manufacturing a porous three-dimensional microfibrous scaffold and a porous three-dimensional microfibrous scaffold manufactured thereby, and more particularly, to a method of manufacturing a porous three-dimensional micro/nanofibrous scaffold, in which a pore size and porosity are easily controlled through an electrohydrodynamic (EHD) process and cell activity is considerably improved, and a porous three-dimensional micro/nanofibrous scaffold manufactured thereby.

2. Discussion of Related Art

Micro/nanofibers have a large surface area, and thus are applied to various bioengineering fields, for example, a filter, a fiber, a photo sensor, drug delivery, a wound dressing, tissue engineering, and biomedicine. Among the various applications, a micro/nanofibrous scaffold has been known to have a form and function allowing the interaction between cells due to a structure similar to an extracellular matrix (ECM) environment, and such a structure plays a very important role in tissue engineering. To form a support having the ECM structure, many researchers use an electrospinning process, and therefore a size of the fiber support can be controlled by controlling an intensity of an electric field, a distance, a nozzle size, and a concentration of a solution (H. Fong et al., Polymer 1999, 40, 4585; P. Gupta et al., Polymer 2005, 46, 4799; L. S. Nair et al., Biomacromolecules 2004, 5, 2212).

However, according to a general process using electrospinning, a two-dimensional fiber is manufactured, and attachment and the interaction of cells only occur on a surface of the two dimensional support. In a human body, cells are interacted in a three-dimensional ECM, and the three-dimensional structure plays a very important role in tissue regeneration (M. W. Kyung et al., J Biomed Mater Res A 2003, 67, 531). According to the conventional electrospinning method, fibers are collected through spinning on a metal plate. To overcome such a shortcoming of the two-dimensional support, in the laboratory of Y. Yokayama, a three-dimensional support was manufactured using a wet electrospinning process (Y. Yokoyama et al., Materials Letters 2009, 63, 754). Other researchers manufactured a three-dimensional fiber support having a height of 1 mm by adding a copper wire ring to the electrospinning process (P. Quynh et al., Biomacromolecules 2006, 7, 2796). In the laboratory of B. Sun, a currently-developed three-dimensional electrospinning process was introduced, but a three-dimensional fiber support which has a thick fiber or enables simultaneous control of a height and a pore structure has not been developed yet (B. Sun et al., Progress in polymer science 2014, 39, 862).

### SUMMARY OF THE INVENTION

The present invention is provided to solve the problems of the conventional art, and as a result of studying a method of manufacturing a scaffold which stably forms a three-dimensional structure and simultaneously improves porosity and a pore size to make cells easily permeate into the scaffold, it was confirmed that a bundle of micro/nano-sized struts having a randomly entangled micro/nanofibrous structure, which are formed in multiple layers, similar to an ECM, is configured by controlling a weight fraction of a polymer, a flow rate (supplying rate) of a polymer solution, a surface tension of target media or a height using an electrohydrodynamic (EHD) jet process. Therefore, based on this, the present invention was completed.

Accordingly, the present invention is directed to providing a method of manufacturing a porous three-dimensional micro/nanofibrous scaffold, which includes: (a) forming a polymer solution by dissolving a polymer in a primary solvent; (b) supplying a voltage to a nozzle spinning the polymer solution; (c) forming an initial jet by discharging the polymer solution from the nozzle; and (d) depositing the discharged initial jet in a bath filled with a secondary solvent. Here, a surface tension of the secondary solvent is smaller than that of the primary solvent.

In addition, the present invention is directed to providing a porous three-dimensional micro/nanofibrous scaffold manufactured by the manufacturing method.

However, the technical objects to be achieved in the present invention are not limited to the above descriptions, and other objects not described herein will be clearly understood by those of ordinary skill in the art from the following descriptions.

In one aspect, the present invention provides a method of manufacturing a porous three-dimensional micro/nanofibrous scaffold, which includes: (a) forming a polymer solution by dissolving a polymer in a primary solvent; (b) supplying a voltage to a nozzle spinning the polymer solution; (c) forming an initial jet by discharging the polymer solution from the nozzle; and (d) depositing the discharged initial jet in a bath filled with a secondary solvent. Here, a surface tension of the secondary solvent is smaller than that of the primary solvent.

In one exemplary embodiment of the present invention, the porous three-dimensional microfiber may have a form of a microfiber, a nanofiber, or a composite thereof.

In another exemplary embodiment of the present invention, in the polymer solution, 8 to 12 wt % of the polymer may be included.

In still another exemplary embodiment of the present invention, the primary solvent may be methylene chloride, dimethyl formamide, or a mixture thereof.

In yet another exemplary embodiment of the present invention, the polymer may be selected from the group consisting of polylactide, polyglycolide, polycaprolactone, polytrimethylenecarborenecarbonate, polyamino acid, polyorthoester, polyethyleneoxide, and a copolymer thereof.

In yet another exemplary embodiment of the present invention, in operation (b), 10 to 14 kV of voltage may be supplied.

In yet another exemplary embodiment of the present invention, in operation (c), the polymer solution may be supplied to the nozzle at a rate of 0.1 to 0.2 ml/h.

In yet another exemplary embodiment of the present invention, the secondary solvent may be ethanol.

In yet another exemplary embodiment of the present invention, the secondary solvent may fill the bath to a height of 4 to 8 mm.

In another aspect, the present invention provides a porous three-dimensional microfibrous scaffold manufactured by the above method.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1A shows a process of manufacturing a three-dimensional micro/nanofibrous scaffold through electrospinning in an ethanol (EtOH) bath;

FIG. 1B shows an SEM image of a scaffold manufactured using a copper plate as a target medium;

FIG. 1C shows an SEM image of a scaffold using water as a target medium;

FIG. 1D shows an SEM image of a scaffold using ethanol as a target medium;

FIG. 1E shows a result confirming the formation of an initial jet according to supplied voltages (7 kV);

FIG. 1F shows a result confirming the formation of an initial jet according to supplied voltages (9 kV);

FIG. 1G shows a result confirming the formation of an initial jet according to supplied voltages (10 kV);

FIG. 1H shows a result confirming the formation of an initial jet according to supplied voltages (14 kV);

FIG. 1I shows a result confirming the formation of an initial jet according to supplied voltages (15 kV);

FIG. 2 shows an SEM image of a three-dimensional micro/nanofibrous scaffold manufactured using PEO;

FIG. 3A shows an SEM image confirming the formation of a three-dimensional micro/nanofibrous structure according to a surface tension;

FIG. 3B shows a result obtained by measuring a change in the diameter of the three-dimensional micro/nanofibrous structure according to the surface tension;

FIG. 4A shows an SEM image confirming the formation of a three-dimensional micro/nanofibrous structure according to a height of target media in a bath;

FIG. 4B shows a result obtained by measuring a change in the diameter of the three-dimensional micro/nanofibrous structure according to the height of the target media in the bath;

FIG. 5A shows an SEM image confirming the formation of a three-dimensional micro/nanofibrous structure according to a supplying rate of a polymer solution;

FIG. 5B shows a result obtained by measuring a change in diameter of the three-dimensional micro/nanofibrous structure according to the supplying rate of the polymer solution;

FIG. 6A shows an SEM image confirming the formation of a three-dimensional micro/nanofibrous structure according to a weight fraction of a polymer;

FIG. 6B shows a result obtained by measuring a change in the diameter of the three-dimensional micro/nanofibrous structure according to the weight fraction of the polymer;

FIG. 7A shows a SEM image of scaffolds having various pore sizes (1 mm) and porosities (93.3%±0.5);

FIG. 7B shows a SEM image of scaffolds having various pore sizes (1.5 mm) and porosities (94.8±0.4);

FIG. 7C shows a SEM image of scaffolds having various pore sizes (2 mm) and porosities (96.7±0.2);

FIG. 8A shows an SEM image of a solid-freeform fabricated scaffold manufactured as a control scaffold;

FIG. 8B shows an SEM image of the scaffold (porosity: 93%) manufactured by a manufacturing method according to the present invention;

FIG. 9A show results confirming a form of MC3T3-E1 (pre-osteoblast) and cell activity of the control scaffold through microscopy after fluorescent staining (diamidino-2-phenylindole (DAPI) and phalloidin) in Example 10;

FIG. 9B show results confirming a form of MC3T3-E1 (pre-osteoblast) and cell activity of the scaffold according to the present invention through microscopy after fluorescent staining (diamidino-2-phenylindole (DAPI) and phalloidin) in Example 10;

FIG. 9C shows results obtained by measuring a cell number per mm² after four hours and 1 day of cell culture with respect to the control scaffold and the scaffold according to the present invention;

FIG. 9D shows results obtained by measuring a proliferation rate after four hours and 1 day of cell culture with respect to the control scaffold and the scaffold according to the present invention;

FIG. 9E shows results obtained by measuring an F-action area after four hours and 1 day of cell culture with respect to the control scaffold and the scaffold according to the present invention;

FIG. 10A shows an SEM image of an electrospun fiber mat;

FIG. 10B shows a result obtained by measuring porosities and fiber diameters of a fiber mat and a scaffold according to the present invention;

FIG. 10C shows a result of comparison in protein absorption performance between the fiber mat and the scaffold according to the present invention;

FIG. 10D shows results confirming cell activities on the fiber mat through microscopy after fluorescent staining (DAPI and phalloidin) in Example 10;

FIG. 10E shows results confirming cell activities on the scaffold according to the present invention through microscopy after fluorescent staining (DAPI and phalloidin) in Example 10; and

FIG. 10F shows a result obtained by measuring cell numbers on surfaces and cross-sections of the fiber mat and the scaffold according to the present invention.

### DETAILED DESCRIPTION OF EXEMPLARY EMBODIMENTS

Hereinafter, the present invention will be described in detail.

In one aspect of the present invention, the present invention provides a method of manufacturing a porous three-dimensional micro/nanofibrous scaffold, which includes: (a) forming a polymer solution by dissolving a polymer in a primary solvent; (b) supplying a voltage to a nozzle spinning the polymer solution; (c) forming an initial jet by discharging the polymer solution from the nozzle; and (d) depositing the discharged initial jet in a bath filled with a secondary solvent. Here, a surface tension of the secondary solvent is smaller than that of the primary solvent. Here, the porous three-dimensional microfiber may have a form of a microfiber, nanofiber, or a composite structure of micro and nanofibers.

According to the present invention, a porous three-dimensional micro/nanofibrous scaffold, in which a pore structure can be controlled by a process of phase changing an initial jet manufactured through an EHD process from a gas phase to a liquid phase using a liquid collector having a low surface tension, may be manufactured.

In operation (a), a polymer solution may be formed by dissolving a polymer in a primary solvent, and the polymer solution preferably includes 8 to 12 wt % of the polymer, and a micro/nanofibrous scaffold manufactured thereby may have and maintain a stable three-dimensional structure.

In one exemplary embodiment of the present invention, as a result of confirming stability of the three-dimensional structure of the nanofibrous scaffold according to a weight fraction of the polymer, it was confirmed that, when 8 to 12 wt % of the polymer is used, the micro/nanofibrous structure was stably formed from a single jet, but when the content of the polymer is more than 12 wt %, an unstable fiber structure was shown (refer to Example 8).

The polymer used in operation (a) may be a biocompatible or biodegradable polymer, and for example, may be, but is not limited to, selected from the group consisting of polylactide, polyglycolide, polycaprolactone, polytrimethylenecarborenecarbonate, polyamino acid, polyorthoester, polyethyleneoxide, and a copolymer thereof. In addition, the primary solvent used in operation (a) may easily dissolve the polymer, and is preferably an organic solvent, more preferably, methylene chloride, dimethyl formamide, or a mixed solvent thereof, and most preferably, a mixed solvent of methylene chloride and dimethyl formamide.

In operation (b), a voltage is supplied to a nozzle spinning the polymer solution formed in operation (a), and 10 to 14 kV of the voltage may be supplied.

In another exemplary embodiment of the present invention, as a result of confirming the formation of an initial jet according to the supplied voltage, it was confirmed that the initial jet was stably formed at 10 to 14 kV (refer to Example 4).

In operation (c), the initial jet is formed by discharging the polymer solution from the nozzle, and here, the polymer solution may be supplied to the nozzle at a rate of 0.1 to 0.2 ml/h.

In still another exemplary embodiment of the present invention, as a result of confirming the formation of the three-dimensional micro/nanofibrous structure according to a supplying rate of the polymer solution, it was confirmed that, when the polymer solution is supplied at a rate of less than 0.2 ml/h, an entangled micro/nanofibrous structure was formed (refer to Example 7).

In operation (d), the initial jet discharged by operation (c) is deposited in a bath filled with a secondary solvent, and here, the secondary solvent may be a solvent having a smaller surface tension than that of the primary solvent. For example, the secondary solvent may be, but is not limited to, ethanol, or any solvent satisfying the above-described condition. In addition, the secondary solvent may fill the bath to a height of 4 to 8 mm.

In yet another exemplary embodiment of the present invention, as a result of confirming the formation of the three-dimensional micro/nanofibrous structure according to the surface tension, it was confirmed that, when a solvent having a lower surface tension than that of the polymer solution is used, the single jet was completely changed into an entangled micro/nanofiber in target media (refer to Example 5).

In yet another exemplary embodiment of the present invention, as a result of confirming the formation of the three-dimensional micro/nanofibrous structure according to a height of the target media in the bath, it was confirmed that when the height is more than 4 mm, an entangled micro/nanofibrous structure was formed (refer to Example 6).

According to the manufacturing method of the present invention, scaffolds having various pore sizes and porosities may be manufactured, and cell activities in the manufactured scaffolds are considerably higher than that of the conventional scaffold.

Therefore, in another aspect of the present invention, the present invention provides a porous three-dimensional micro/nanofibrous scaffold manufactured by the manufacturing method.

The term “cell” used herein refers to a cell used in regeneration of cells or tissues, and has a wide meaning including a cell differentiating into various cells, such as a stem cell and a monocyte cell, as well as various adult cells including an epidermal cell, a vascular cell, an osteocyte, an osteoblast, and a chondrocyte.

In yet another exemplary embodiment of the present invention, as a result of comparing cell activity between the scaffold manufactured by the manufacturing method of the present invention and the conventional scaffold, that is, between a solid-freeform fabricated scaffold and a fiber mat, it was confirmed that the cell activity of the scaffold manufactured by the manufacturing method of the present invention was considerably improved (refer to Examples 10 and 11).

Hereinafter, exemplary examples will be provided to help in understanding the present invention. However, the following examples are merely provided to more easily understand the present invention, but the scope of the present invention is not limited thereto.

[Materials]

Poly(ε-caprolactone) (hereinafter, referred to as “PCL,” concentration: 1.135 g/cm³, molecular weight (Mₙ): 90,000 g/mol, melting point: 60° C.) and polyethyleneoxide (hereinafter, referred to as “PEO,” Mₙ: 900,000 g/mol) were obtained from Sigma-Aldrich Co. LLC. (St. Louis, Mo., USA). Methylene chloride (hereinafter, referred to as “MC”; surface tension: 28.1 mN/m) and dimethyl formamide (hereinafter, referred to as “DMF”; surface tension: 37.1 mN/m) were obtained from Junsei Chemical Co. Ltd.(Tokyo, Japan), and a 16-G electrospinning nozzle and 20-ml glass syringe were used for electrospinning. As solvents (used as target media) added in the bath, 99% ethanol (EtOH; surface tension: 22.1 mN/m; Duksan, South Korea) and water (surface tension: 72.9 mN/m) were used.

### EXAMPLES

### Example 1

### Experiment Method

1-1. In vitro Cell Culture

A scaffold (5 mm×5 mm) was sterilized with 70% ethanol and ultraviolet (UV) rays and put into a culture medium overnight. To evaluate behaviors of cells cultured in the scaffold, MC3T3-E1 cells (Mouse pre-osteoblast cells; ATCC, Manassas, Va., USA) were used. The MC3T3-E1 cells were cultured for 4 passages in a 24-well plates containing an a-minimum essential medium (Life Sciences, USA) supplemented by 10% fetal bovine serum (Gemini Bio-Products, USA) and 1% antibiotic/antimycotic solution (Cellgro, USA). Afterward, the cells were collected by trypsin-ethylenediaminetetraacetic acid (EDTA), inoculated into the scaffold at a density of 1×10⁵ per sample, and cultured at 37° C. under 5% CO₂. After 4 hours, 1 day and 3 days of cell culturing, DAPI fluorescent staining was performed to distinguish a cell nucleus in the scaffold. In addition, to visualize an actin cytoskeleton of proliferated cells, phalloidin staining (Invitrogen, Carlsbad, Calif.) was performed. A cell number confirmed by the DAPI staining was measured from fluorescent images of a surface and a cross-section of the scaffold using Image J. The stained scaffold was analyzed using an epifluorescence attachment and microscope (TE2000-S; Nikon, Tokyo, Japan) equipped with an SPOT RT digital camera (SPOT Imaging Solutions, Sterling Heights, Mich., USA).

1-2. Statistical Analysis

All of the proposed data are expressed as average±SD. Statistical analysis was performed using an SPSS software (ver. 20.0; SPSS Inc.), and performed through single-factor analyses of variance (ANOVA). A significant level was set at p <0.05, and “NS” denoted non-significance.

### Example 2

### Manufacture of Three-Dimensional Micro/Nanofibrous Scaffold Using PCL

2-1. Use of Ethanol as Target Media

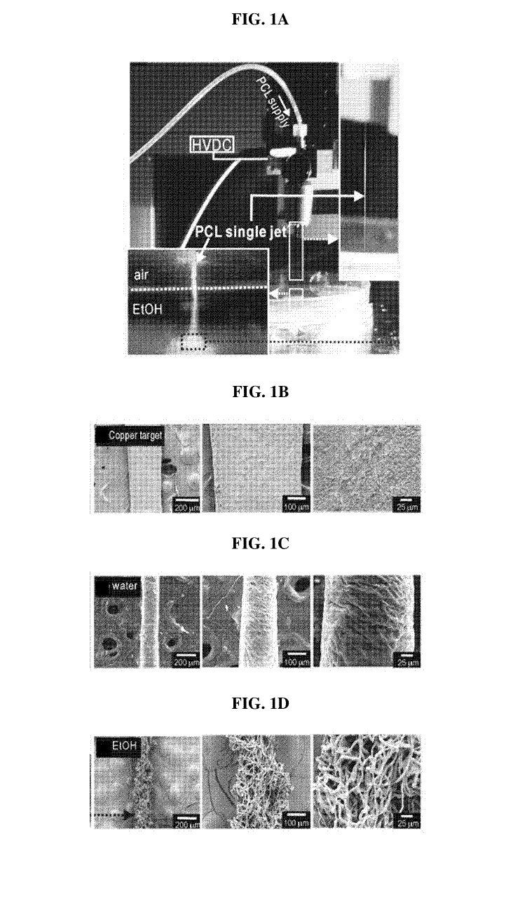

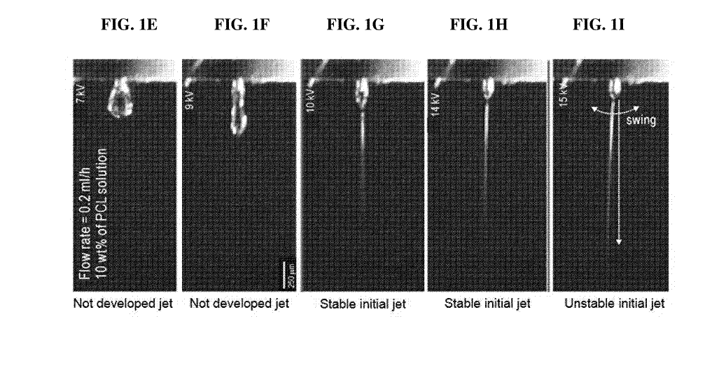

A three-dimensional micro/nanofibrous scaffold was manufactured by dissolving PCL in a mixed solvent prepared by mixing MC and DMF at a ratio of 80:20 and electrospinning the resulting solution, and FIG. 1(a) schematically illustrates a process of manufacturing a three-dimensional micro/nanofibrous scaffold by performing electrospinning in an ethanol bath (EtOH bath).

That is, as shown in FIG. 1(a), an initial jet was formed by discharging the PCL-mixed solution from a nozzle while supplying an electric field, the initial jet was deposited in the ethanol bath (target media), and distributed by a triaxial robot system.

Here, the PCL was included at 10 wt % in the PCL solution, and a supplied electric field and a flow rate (supplying rate) were 1.9 kV/cm (11 kV) and 0.2 ml/h, respectively. To manufacture a fibrous scaffold, an electrospinning system was coupled to the triaxial robot (DTR3-2210-T-SG, DASA Robot, South Korea), and automatically moved according to a CAD model. Here, a moving speed of the nozzle was 10 mm/s Ethanol (EtOH) media were used as a target, and put into a grounded bath containing a grounded copper plate. The flow rate of the PCL solution was controlled using a syringe pump (KDS 230; KD Scientific, Holliston, Mass., USA), and a power supply (SHV300RD-50K; Convertech, Seoul, South Korea) was used to generate an electric field. During electrospinning, a temperature was 28° C., and humidity was 36±3%.

After the three-dimensional fibrous scaffold was manufactured, ethanol (EtOH) was removed using water (triple-distilled water), and the scaffold was vacuum freeze dried at −76° C. for one day using a freeze drier (SFDSM06; Samwon, Busan, South Korea). Then, the dried result was sputter-coated with gold.

2-2. Use of Copper Plate as Target Media

A scaffold was manufactured by the same method as described in Example 2-1, except that a copper plate was used as the target media.

2-3. Use of Water as Target Media

A scaffold was manufactured by the same method as described in Example 2-1, except that water was used as the target media.

2-4. Confirmation of Scaffold Structure

Structures of the scaffolds manufactured in Examples 2-1 to 2-3 were confirmed through scanning electron microscopy (SEM; SNE-3000M, SEC, Inc., South Korea), and the results are shown in FIGS. 1(b) to 1(d), respectively.

When the cooper plate was used as the target media, as shown in FIG. 1(b), the initial jet was deposited on a target plate, and a deposited form was identified as a pressed single micro-sized strut. In addition, when water was used as the target media, as shown in FIG. 1(c), the manufactured scaffold had a cylindrical form, and it is assumed that this is because water serves as an elastic cushion with respect to a single jet (Ahn, S. H.; Lee, H. J.; Kim, G. H. Polycaprolactone Scaffolds Fabricated with an Advanced Electrohydrodynamic Direct-Printing Method for Bone Tissue Regeneration. Biomacromolecules 2011, 12 (12), 4256-4263).

However, as shown in FIG. 1(d), it was confirmed that, when ethanol was used as the target media according to the present invention, a single jet was minutely divided into an entangled micro/nanofibrous bundle (average diameter of fiber: 3.2±0.7 μm) due to a charged single jet of a PCL solution contained in ethanol media.

### Example 3

### Manufacture of Three-Dimensional Micro/Nanofibrous Scaffold Using PEO

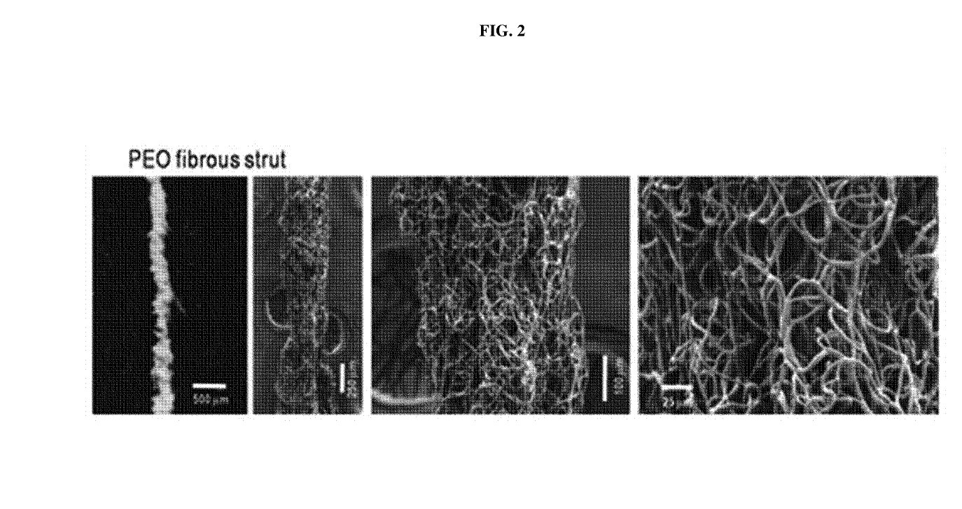

A three-dimensional micro/nanofibrous scaffold was manufactured using PEO as a polymer, which was included at 5 wt % in a PEO solution, and a supplied electric field and a flow rate (supplying rate) were 1.9 kV/cm (11 kV) and 2.5 ml/h, respectively, and an ethanol height in a bath was 4 mm. A structure of the three-dimensional micro/nanofibrous scaffold was confirmed by SEM, and the result is shown in FIG. 2.

As shown in FIG. 2, it was confirmed that a fiber strut of PEO was easily made.

### Example 4

### Confirmation of Formation of Initial Jet According to Supplied Voltage

An initial jet was formed by the same method as described in Example 2-1, except that various voltages (7, 9, 10, 14, 15 kV) were supplied, and the results are shown in FIGS. 1(e) to 1(i), respectively.

As shown in FIGS. 1(e) to 1(i), it was confirmed that the initial jet was not formed at less than 10 kV, and the initial jet was unstably formed at more than 14 kV. However, it was confirmed that the initial jet was stably formed at 10 to 14 kV.

### Example 5

### Confirmation of Formation of Three-Dimensional Micro/Nanofibrous Structure According to Surface Tension

Generally, in an electrospinning process, critical material parameters to manufacture a micro/nanofiber include electric conductivity, a solution surface tension, viscosity, etc. When a supplied electric field reaches a critical voltage, a Taylor cone of a nozzle tip overcomes the surface tension of a solution emitted into the electric field, and a single solution jet may be formed (refer to FIG. 1(g)). The single initial jet may break up to the micro/nanofiber due to three types of continuous bending instability. A final diameter of a liquid charged in the electric field having bending instability may be estimated using the following formula by Fridrikh et al. (Fridrikh, S. V.; Yu, J. H.; Brenner, M. P.; Rutledge, G. C. Controlling the fiber diameter during electrospinning Physical review letters 2003, 90 (14), 144502-144502.).

0.64γεQ²/[I²(2 lnχ−3) ]^{1/3}

Here, γ is a surface tension of the solution, ε is a dielectric constant of a medium surrounding the jet, Q is a flow rate, I is a current, and χ is a dimensionless wavelength of the bending instability.

From the above formula, it is seen that the surface tension of the solution is one of the critical parameters having an influence on the final diameter of the micro/nanofiber. A relationship between the fiber diameter and the surface tension of the solution may be simply described. That is, when a constant electric field is supplied to the polymer solution and the surface tension of the solution is low, the break-up of the single initial jet may be more rapidly obtained from the nozzle tip.

For this reason, to confirm the surface tension effect of target media in the formation of a micro/nanofibrous structure from the single jet, various mixtures of water and ethanol were used in a target bath.

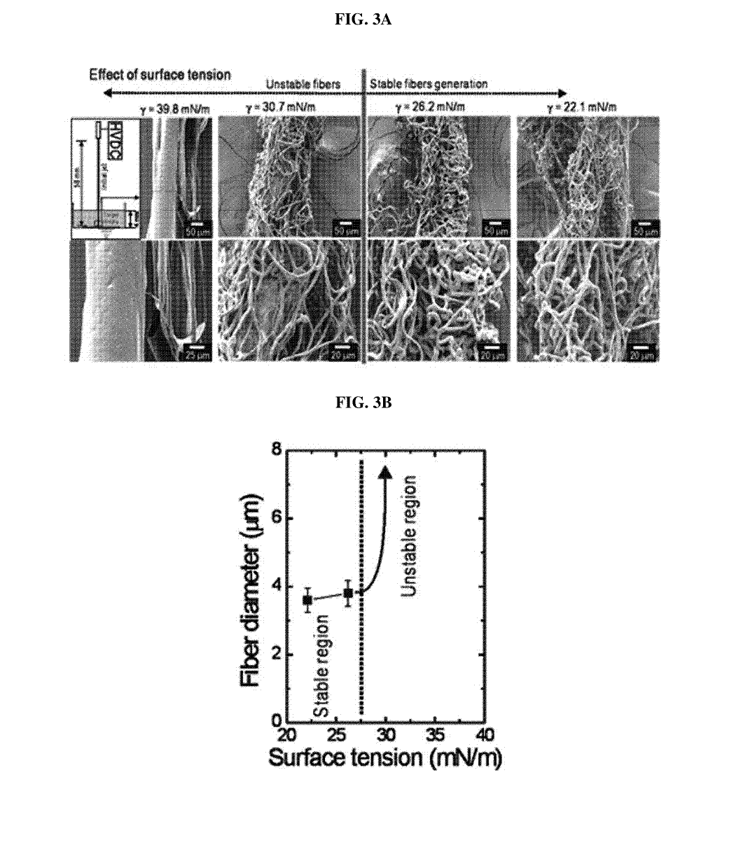

That is, four types of mixed solvents having different surface tensions (22.1 mN/m, 26.2 mN/m, 30.7 mN/m, and 39.8 mN/m) were obtained from the target bath using a simple mixing rule for various mixing ratios of water (surface tension: 72.8 mN/m) and ethanol (surface tension: 22.1 mN/m). The micro/nanofibrous structure was formed by the same method as described in Example 2-1, except that each mixed solvent was used as target media, and the structure was confirmed using SEM, and a diameter of the formed fiber was measured at the same time. The results are shown in FIGS. 3(a) and 3(b), respectively.

As shown in FIGS. 3(a) and 3(b), the single jet was deposited in a form of a single micro-sized strut (diameter: 123±26 μm) in the target media having a surface tension of 39.8 mN/m, and it was confirmed that, as the surface tension of the target media was reduced, the initial jet was changed into an entangled micro/nanofiber. Particularly, at a surface tension of 26.2 mN/m or less, the single jet was perfectly changed into the entangled micro/nanofiber (diameter: 3.3±1.2 μm) in the target media.

From the above results, when the surface tension of the target media contained in the bath was lower than that of the solvent of the PCL solution (surface tension: 29.8 mN/m), it is seen that the entangled micro/nanofibrous structure was formed, and it is assumed that this is because the micro/nanofibrous structure was formed by overcoming the surface tension due to rapid mass exchange and a surface charge inhibited by a surface tension of a MC and DMF mixture when the single jet was put into the target media in the bath.

### Example 6

### Confirmation of Formation of Three-Dimensional Micro/Nanofibrous Structure According to Height of Target Media in Bath

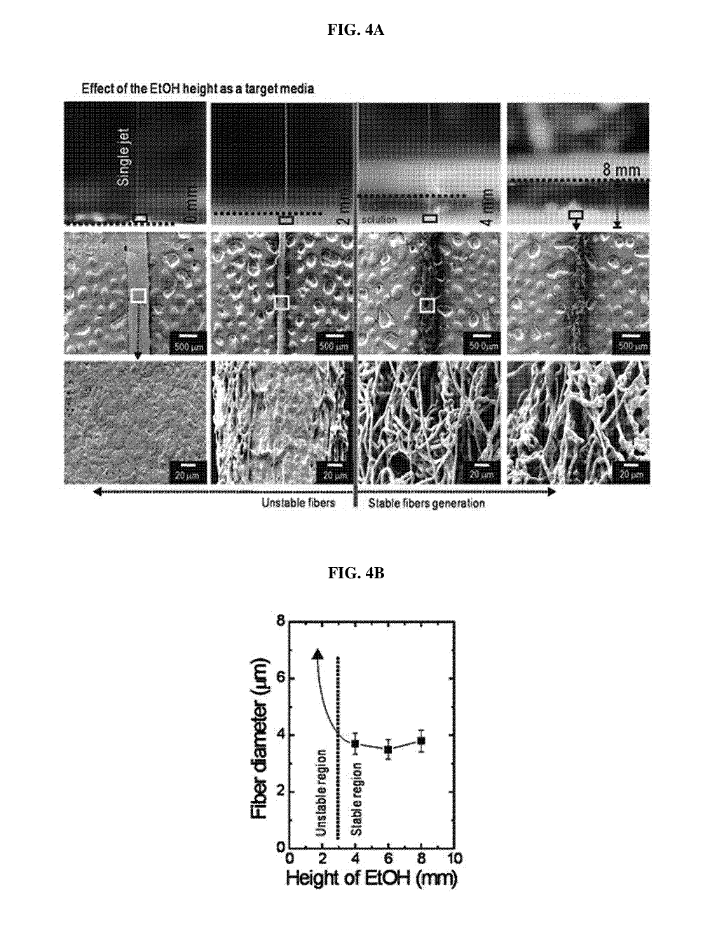

To confirm the formation of a three-dimensional micro/nanofibrous structure according to a height of a target media, that is, ethanol (EtOH) in a bath, the micro/nanofibrous structure was formed by the same method as described in Example 2-1, except that ethanol filled the bath to various heights (0, 2, 4, 8 mm), and the structure was confirmed using SEM and a diameter of the formed fiber was measured at the same time. The results are shown in FIGS. 4(a) and 4(b), respectively.

As shown in FIGS. 4(a) and 4(b), it was confirmed that, in the case of an ethanol-free media, the single jet was deposited in the form of a single strut on a target plate, and as a height of the ethanol media in the bath is increased, the single jet is changed into a mixture of a micro-sized strut and a unstably formed fiber. Particularly, it was confirmed that when the height of the ethanol was more than 4 mm, the entangled micro/nanofibrous structure was formed.

The effect of the height of ethanol in the bath in the formation of the micro/nanofibrous structure may be described by a mass transfer rate between the solvents (MC and DMF) and ethanol. That is, this phenomenon may be described by solvent diffusivity from a cylindrical single jet according to the Fick's second law.

$$\frac{1}{r} [ \frac{\partial }{\partial r} ( {D}_{MS} \cdot r \frac{\partial {C}_{MS}}{\partial x} ) ] = \frac{\partial {C}_{MS}}{\partial t}$$

Here, D_{MS} and C_{MS} are diffusion coefficients of the mixed solvent in the single jet and concentrations of a remnant solvent at the positions of the cylindrical jet (r and x), and t is diffusion time.

From the above formula, on a surface of the cylindrical jet, the solvents (MC and DMF) in the target media were continuously exchanged with ethanol until a concentration of the solvent in the cylindrical jet was in equilibrium with a concentration of the solvent in the target bath. However, when the height of the ethanol was less than 2 mm, the exchange between the solvent and the ethanol was restricted to a limited area of the media resulting in a slow exchange between the solvent and the ethanol only on the surface of the single jet (refer to FIG. 4).

### Example 7

### Confirmation of Formation of Three-Dimensional Micro/Nanofibrous Structure According to Supplying Rate of Polymer Solution

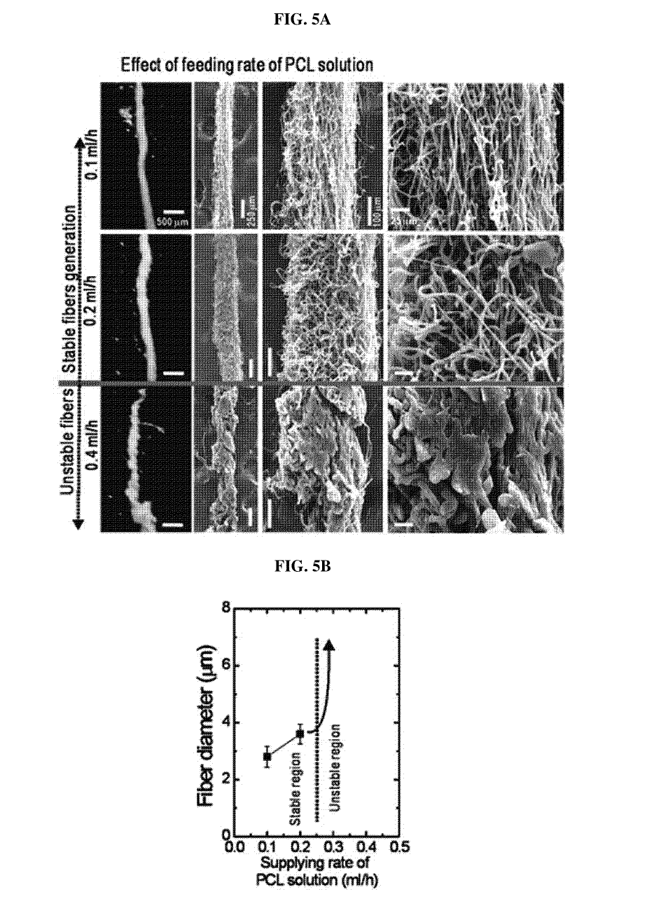

To confirm the formation of a three-dimensional micro/nanofibrous structure according to a supplying rate of a polymer solution, that is, a supplying rate of a PCL solution, the micro/nanofibrous structure was formed by the same method as described in Example 2-1, except that the PCL solution was provided at various supplying rates (0.1, 0.2, 0.4 ml/h), and the structure was confirmed using SEM and a diameter of the formed fiber was measured at the same time, and the results are shown in FIGS. 5(a) and 5(b), respectively. Meanwhile, a height of the ethanol was set to 4 mm.

As shown in FIGS. 5(a) and 5(b), it could be confirmed that the supplying rate of the PCL solution had an influence on formation of the fiber structure. That is, when the supplying rate was less than 0.2 m/h, an entangled micro/nanofibrous structure was formed, but when the supplying rate was more than 0.2 m/h, it was confirmed that, due to an insufficient mass transfer time between the solvent of the PCL solution and the ethanol in the target bath, the fiber structure was unstably formed from the micro-sized single jet.

### Example 8

### Confirmation of Formation of Three-Dimensional Micro/Nanofibrous Structure According to Weight Fraction of Polymer

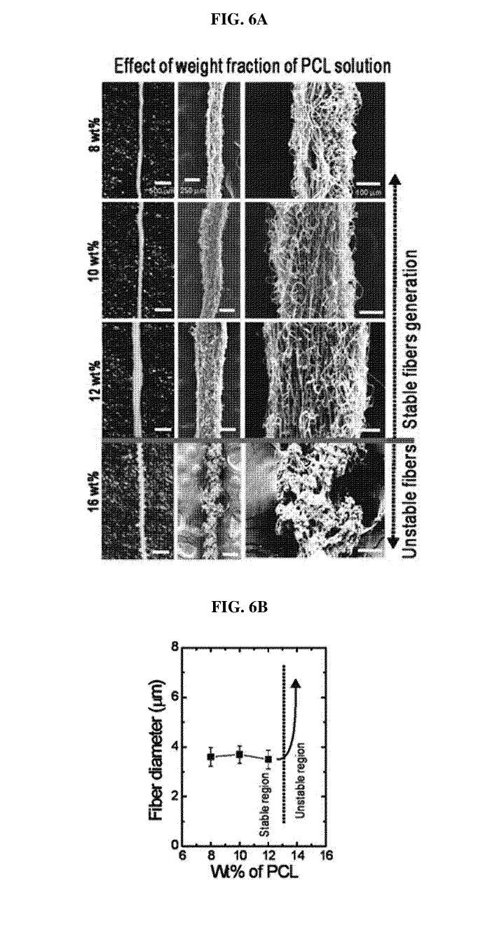

To confirm an influence of a weight fraction of a polymer, that is, PCL, on the formation of a three-dimensional micro/nanofibrous structure, the micro/nanofibrous structure was formed by the same method as described in Example 2-1, except that PCL having various weight fractions (8, 10, 12, and 16 wt %) was used, and the structure was confirmed using SEM and a diameter of the formed fiber was measured at the same time, and the results are shown in FIGS. 6(a) and 6(b), respectively.

As shown in FIGS. 6(a) and 6(b), it was confirmed that, as the weight fraction was increased from 8 wt % to 12 wt %, the micro/nanofibrous structure was stably formed from the single jet. However, it was confirmed that, when the weight fraction is more than 12 wt %, the unstable fiber structure was shown, and it is understood that this is because a repulsive force generated from the surface charge did not overcome a relatively high viscosity of the PCL solution.

### Example 9

### Manufacture of Three-Dimensional Micro/Nanofibrous Scaffold Having Various Pore Sizes and Porosities

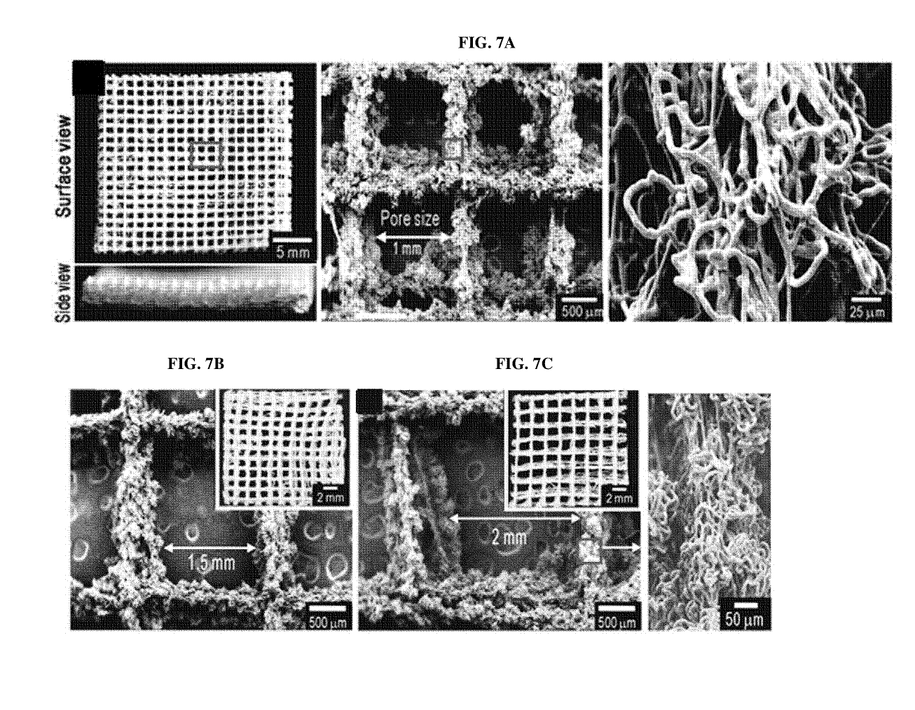

Three-dimensional micro/nanofibrous scaffolds having various pore sizes and porosities were manufactured under stable process conditions confirmed through Examples 4 to 8, that is, by setting a concentration of PCL in the PCL solution to 10 wt % (MC: 72 wt %, DMF: 18 wt %) using pure ethanol as a target media at an electric field of 1.9 kV/cm, a PCL supplying rate of 0.2 ml/h, a height of ethanol in a target bath of 4 mm, and a nozzle speed of 10 mm/s, structures of the scaffolds were confirmed through SEM, and the results are shown in FIGS. 7(a) to 7(c).

More specifically, it was confirmed that, as shown in FIGS. 7(a) to 7(c), scaffolds having various pore sizes (1, 1.5, and 2 mm) and porosities (93.3%±0.5, 94.8±0.4, 96.7±0.2) can be manufactured by the manufacturing method according to the present invention, and in the manufactured scaffolds, struts having a distribution width of 324±75 μm formed a layer-by-layer structure.

### Example 10

### Confirmation of Cell Activity of Three-Dimensional Micro/Nanofibrous Scaffold

10-1. Manufacture of Control Scaffold

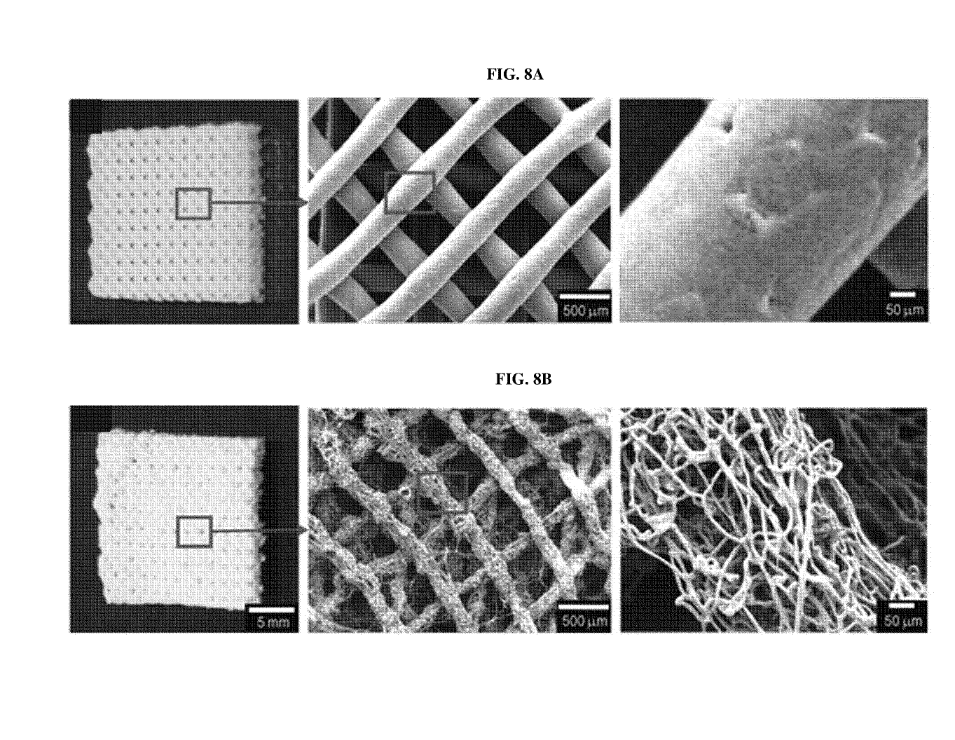

To compare cell activity of the scaffold manufactured by the manufacturing method according to the present invention, first, a solid-freeform fabricated scaffold having a similar geometrical structure (porosity: 59%) was manufactured. More specifically, PCL powder was transferred to a heating barrel (110° C.) of a plotting system. To stably attach struts of the layer, the struts extruded onto a previous layer were slightly pressed, and a gap between the nozzle tip (diameter=250 μm) and the previous layer was set to approximately 92%, compared to diameters of the extruded struts. A control scaffold was manufactured by setting a transfer rate and an extruding pneumatic pressure of a plotter to 3 mm/s and 673±31 kPa, respectively, and an SEM image of the control group is shown in FIG. 8(a).

Meanwhile, an SEM image of the scaffold (porosity: 93%) manufactured by the manufacturing method according to the present invention to confirm cell activity is shown in FIG. 8(b).

10-2. Comparison of Activities in Scaffolds

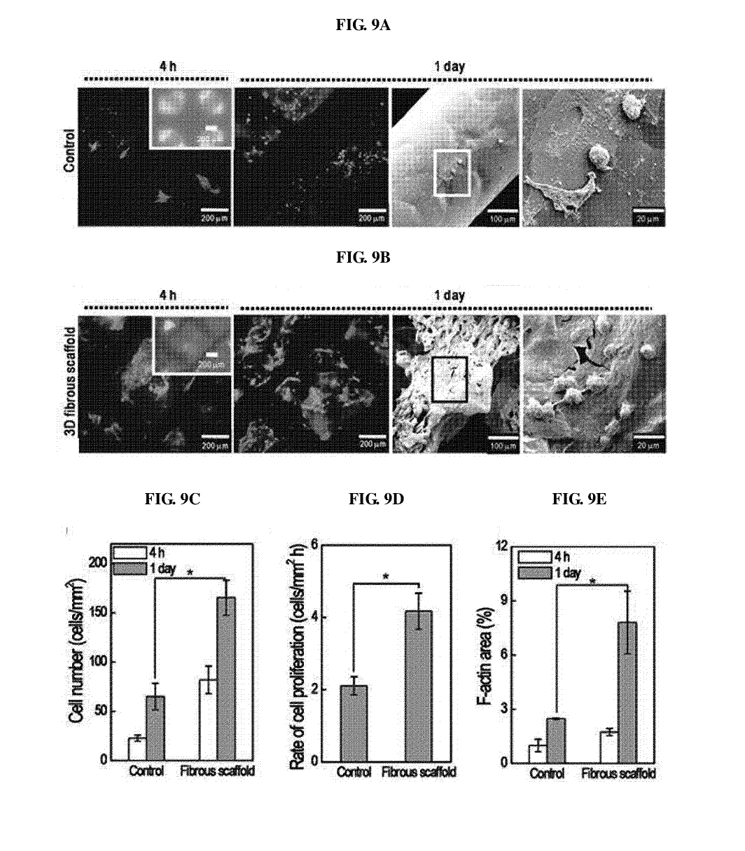

To confirm the form of MC3T3-E1 (pre-osteoblast) and cell activity, the control scaffold and the scaffold according to the present invention were observed through fluorescent staining (DAPI and phalloidin) and microscopy as described in Example 1-1, and the results are shown in FIGS. 9(a) and 9(b).

Since the micro/nanofiber had a considerably large influence on initial cell attachment and proliferation, according to the observation of the activities of the scaffolds after 4 hours and one day of cell culturing, as shown in FIGS. 9(a) and 9(b), it could be confirmed that initial cell-attachment more highly occurred, and a cytoskeleton was well formed on the scaffold according to the present invention, compared to the control scaffold (nucleus: blue, F-actin: red).

Additionally, according to the result of DAPI/phalloidin staining analyzed by Image J software (National Institutes of Health, Bethesda, Md., USA), a cell number per mm², a proliferation rate (a gradient between a cell number vs. a culture time), and an F-action area were measured after 4 hours and 1 day of cell culturing, and the results are shown in FIGS. 9(c) to 9(e), respectively. As shown in FIGS. 9(c) to 9(e), it was confirmed that, compared to the control scaffold, cells of the scaffold of the present invention showed considerably high initial cell attachment and proliferation. In addition, it was confirmed that the scaffold according to the present invention impressively developed the cytoskeleton of the MC3T3-E1 cell.

### Example 11

### Comparison in Cell Activity with Electrospun Fiber Mat

11-1. Manufacture of Fiber Mat

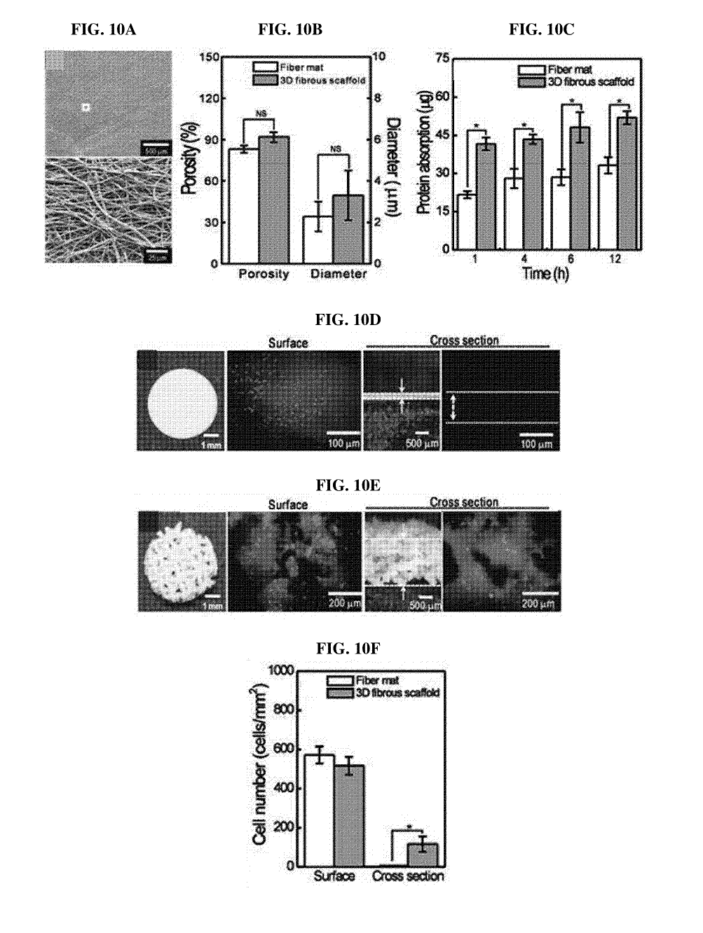

To compare cell activity between a normal electrospun fiber mat and the three-dimensional scaffold according to the present invention, first, a fiber mat having porosity and fiber diameter, which were similar to those of the three-dimensional scaffold according to the present invention, was manufactured using a general electrospinning process (refer to FIGS. 10(a) and 10(b)).

Here, the porosity of the scaffold was calculated using the following formula.

1−M/(ρV)

Here, M is a mass of the scaffold, ρ is the concentration of PCL (1.135 g/cm³), and V is a volume of a structure (assumed as a rectangular form).

11-2. Comparison in Protein Absorption Performance

Generally, protein absorption performance can have a large influence on initial cell attachment, which is caused by absorption of fibronectin and vitronectin. In this example, the following experiment was performed to compare protein absorption performance between the conventional fiber mat and the three-dimensional scaffold according to the present invention.

That is, the protein absorption performance was measured using a bicinchoninic acid protein assay (BCA; Pierce Kit; Thermo Scientific, Waltham, Mass., USA). A sample (diameter=8 mm, weight=3 mg±1.5 mg) was placed on 24-well plates containing a Dulbecco's modified Eagle's medium (DMEM; Hyclone, Logan, Utah, USA) supplemented with 10% fetal bovine serum (Hyclone), and cultured at 37° C. for 1, 4, 6, and 12 hours. Afterward, the sample was washed with PBS, and dissolved using Triton X-100 0.1%. An aliquot (25 μl) of the lysate was added to 200 μl of a BCA agonist, and the mixture was cultured 37° C. for 30 minutes. An absorbance was measured at 562 nm using a plate reader. Here, the sample cultured in a serum-free medium was used as a blank. The protein absorption performance was calculated as the average±standard deviation (n=5), and the result is shown in FIG. 10(c).

As shown in FIG. 10(c), it was confirmed that, compared to the conventional fiber mat, considerably improved protein absorption performance was shown in the scaffold according to the present invention. From the result, it was seen that the scaffold according to the present invention can provide high cell attachment and proliferation, compared to the electrospun mat.

11-3. Comparison Using DAPI/Phalloidin Staining

MC3T3-E1 cells were cultured on a fiber mat and the scaffold according to the present invention, and to confirm cell activity after 3 days of the culturing, the cells were stained with DAPI and phalloidin and observed by microscopy according to the method described in Example 1-1. The results are shown in FIGS. 10(d) and 10(e), respectively. In addition, cell numbers were measured from the DAPI/phalloidin staining using Image J software (National Institutes of Health, Bethesda, Md., USA), and the result is shown in FIG. 10(f).

As shown in FIGS. 10(d) to 10(f), it was confirmed that all of ECM-containing cells were well proliferated on surfaces of the fiber mat and the scaffold according to the present invention (nucleus: blue, F-actin: red), but the cells cultured on the scaffold according to the present invention more easily permeated into the scaffold and proliferated than those on the fiber mat.

According to the present invention, a porous three-dimensional micro/nanofibrous scaffold in which a pore structure can be controlled by a process of phase changing a fiber (initial jet) manufactured through an EHD process from a gas phase to a liquid phase using a liquid collector having a low surface tension can be provided.

While the present invention has been shown and described with reference to certain exemplary embodiments thereof, it will be understood by those skilled in the related art that various changes in form and details may be made therein without departing from the gist and scope of the present invention as defined by the appended claims.

1. A method of manufacturing a porous three-dimensional micro/nanofibrous scaffold, comprising:
(a) forming a polymer solution by dissolving a polymer in a primary solvent;
(b) supplying a voltage to a nozzle spinning the polymer solution;
(c) forming an initial jet by discharging the polymer solution from the nozzle; and
(d) depositing the discharged initial jet into a bath filled with a secondary solvent,
wherein a surface tension of the secondary solvent is smaller than that of the primary solvent.

2. The method according to claim 1, wherein the porous three-dimensional microfiber has a form of a microfiber, a nanofiber or a composite thereof.

3. The method according to claim 1, wherein the polymer is included in the polymer solution at 8 to 12 wt %.

4. The method according to claim 1, wherein the primary solvent is methylene chloride, dimethyl formamide or a mixture thereof.

5. The method according to claim 1, wherein the polymer is selected from the group consisting of polylactide, polyglycolide, polycaprolactone, polytrimethylenecarbonenecarbonate, polyamino acid, polyorthoester, polyethyleneoxide and a copolymer thereof.

6. The method according to claim 1, wherein, in operation (b), the voltage is supplied at 10 to 14 kV.

7. The method according to claim 1, wherein, in operation (c), the polymer solution is supplied to the nozzle at a rate of 0.1 to 0.2 ml/h.

8. The method according to claim 1, wherein the secondary solvent is ethanol.

9. The method according to claim 1, wherein the secondary solvent fills the bath to a height of 4 to 8 mm.

10. A porous three-dimensional micro/nanofibrous scaffold manufactured by the method according claim 1.
